(19) United States
(12) Patent Application Publication
(10) Pub. No.: US 2017/0086778 A1
(43) Pub. Date: Mar. 30, 2017

(54) CAPTURE AND ANALYSIS OF BODY SOUNDS

(71) Applicant: International Business Machines Corporation, Armonk, NY (US)

(72) Inventors: Amos Cahan, Dobbs Ferry, NY (US); Theodore G. van Kessel, Millbrook, NY (US)

(21) Appl. No.: 14/869,280

(22) Filed: Sep. 29, 2015

(51) Int. Cl. A61B 7/02; A61B 7/00; A61B 5/021; A61B 5/0476; A61B 5/0408; A61B 5/00; G10L 25/66; A61B 5/026

(52) U.S. Cl. CPC A61B 7/02; G10L 25/66; A61B 7/008; A61B 5/026; A61B 7/003; A61B 5/0476; A61B 5/0408; A61B 5/0022; A61B 5/021; A61B 5/002; A61B 5/7271; A61B 2505/07

(57) ABSTRACT

One or more body sounds are captured using one or more acoustic sensors placed on one or more locations of a subject. The one or more body sounds are converted into one or more acoustic signals using one or more acoustic transducers. The one or more acoustic signals are transmitted to one or more computing devices. The one or more acoustic signals are compared against one or more other signals. At least one of one or more physiological conditions and one or more pathological conditions are identified based on the comparison.

### BACKGROUND

Monitoring of physiological parameters is an important aspect in evaluating and predicting the health status of patients, as accurate medical diagnosis often relies on efficient distinction between what is considered normal and abnormal. Detection and analysis of sounds from the internal organs and systems of a patient's body is often helpful in assessing the patient's health condition. Auscultation is a diagnostic method commonly used during a physical examination that involves listening to internal sounds of a patient's body. Auscultation can be used to localize an abnormality so as to characterize it, as specific pathologic conditions are associated with distinct patterns of abnormal sounds. The timing of an auscultated sound is important, for example associating a heart murmur with a valve disease. Auscultation may be performed using a stethoscope for the purposes of examining the circulatory and respiratory systems (e.g., listening to heart and breath sounds), as well as the gastrointestinal system (e.g., listening to bowel sounds).

### SUMMARY

Embodiments of the invention provide techniques for capture and analysis of body sounds.

For example, in one embodiment, a method comprises the following steps. One or more body sounds are captured using one or more acoustic sensors placed at one or more locations of a subject. The one or more body sounds are converted into one or more acoustic signals using one or more acoustic transducers. The one or more acoustic signals are transmitted to one or more computing devices. The one or more acoustic signals are compared against one or more other signals. At least one of one or more physiological conditions and one or more pathological conditions are identified based on the comparison.

### BRIEF DESCRIPTION OF THE DRAWINGS

FIG. 1 illustrates an overview process of a methodology for capturing and analyzing body sounds, according to an embodiment of the invention.

FIG. 2 illustrates an exemplary embodiment of a system for implementing the methodology of FIG. 1.

FIG. 3 illustrates an exemplary multi-sensor embodiment of a system for implementing the method of FIG. 1.

FIG. 4 illustrates an exemplary application of the system for capturing and analyzing body sounds, according to an embodiment of the invention.

FIG. 5 illustrates a cloud computing environment, according to an embodiment of the invention.

FIG. 6 depicts abstraction model layers according to an embodiment of the invention.

### DETAILED DESCRIPTION

Illustrative embodiments of the invention may be described herein in the context of illustrative methods, systems and devices for capture and analysis of body sounds. However, it is to be understood that embodiments of the invention are not limited to the illustrative methods, systems and devices but instead are more broadly applicable to other suitable methods, systems and devices.

Monitoring physiological parameters may include using acoustic methods of diagnosis that exploit the audio acoustic nature of signals produced by a subject, e.g., human or animal. These methods allow for accurate detection of normal and/or abnormal cases, such that normal is indicative of a healthy patient, while abnormal is indicative of injury, organ dysfunction, illness or the like.

Analysis of heart, lung and vascular disorders by means of noninvasive auscultation is a useful tool for medical diagnosis of ailments. Auscultation generally refers to methods of listening to internal sounds of the body. Auscultation may be performed by listening through a stethoscope, where a medical professional may auscultate a patient's lungs, heart, and intestines to evaluate the timing, frequency, intensity, duration, number, or quality of sounds.

For example, sounds produced by breathing, coughing and more generally lung sounds may be used to determine the physiology or pathology of lungs and reveal cases of airway obstruction or pulmonary disease. As another example, auscultation to the heart sounds may be an important diagnostic clinical tool. Abnormal heart sounds may represent rhythm disorders and/or abnormal heart function due to pressure overload, volume overload, abnormal valve opening or closure and paravalvular leak.

Similarly, auscultation to sounds emitted by the flow of blood through blood vessels may provide clues to the presence of stenosis, aneurysm, fistula, arteriovenous malformation, hemangioma, perivascular space occupying lesion and other abnormalities. This practice relies on the fact that laminar blood flow is disrupted by changes in the internal diameter of a vessel or in its cross-sectional structure, resulting in turbulent blood flow. Turbulent blood flow is associated with an altered pattern of acoustic waves generated.

Auscultation to sounds emitted around the abdomen area may provide information regarding the state of a patient's digestive system. For example, bowel sounds may be abnormal when there is a mechanical problem (e.g., stricture, fistula, obstruction), a neural abnormality (e.g., irritable bowel syndrome) or a vascular problem (e.g., abdominal angina).

Traditional auscultation to body sounds is performed by a medical caregiver (usually a physician) by placing the diaphragm of a stethoscope over the body area to be auscultated. However, traditional auscultation methods are limited in its capacity to collect and interpret body sounds in multiple ways:
(a) Auscultation is generally limited to audible sound, with ultra-sound and infrasound remaining uncaptured.
(b) Traditional auscultation typically lasts seconds to one minute. It therefore captures body sounds at the point of care, at one point in time when the patient is at rest. It is thereby impossible to capture body sounds for prolonged periods of times under varying levels of activity and in various locations using traditional auscultation.
(c) Interpretation of body sounds captured with traditional auscultation is done by the auscultating caregiver using their subjective judgment. The sounds heard by the caregiver are not sharable with others and so, are usually not corroborated by others in real time. Studies have shown poor between-physician concordance in interpreting heart sounds and cervical bruits.
(d) Temporal comparison between body sounds and other patient data such as electrocardiogram (EKG), electroencephalogram (EEG), pulse oximetry, exhaled and blood carbon dioxide (CO₂) level is technically very difficult using traditional auscultation methods.

Advantageously, some embodiments provide means to more efficiently capture, store, share and interpret body sounds. Some embodiments herein also enable prolonged, intermittent or continuous monitoring of body sounds at different body locations, and in different settings (e.g., patient's home) without interfering with a patient's normal activities. Some embodiments also enable capturing body sounds of a patient when a physician is not present, e.g., when telemedicine is used. Some embodiments described herein also provide methods to objectively analyze data relating to body sounds, taking into account the patient's body sounds captured at different points in time and comparing them to data captured from other body parts and other monitored information such as physical activity (e.g., position, steps), EKG, EEG and pulse oximetry. This will enable new insights to be leveraged that could improve diagnostic tools for use in medicine.

Some embodiments provide methods of listening to body sounds with the aim of collecting data and associating the data with diagnostic outcomes using machine learning methods. Some embodiments further include the fusion and synchronization of acoustic data with other data sources, including but not limited to, EKG, EEG, accelerometer, etc. Some embodiments provide methods and systems comprising at least one acoustic transducer, an analog to digital conversion device, and microprocessor, and a mode of transmitting and storing data on local and remote servers. Some embodiments enable collection of acoustic data over an extended period of time.

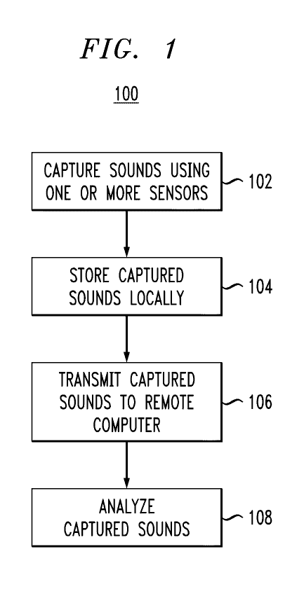

Referring to the figures, FIG. 1 depicts an overview of a method for capturing and analyzing body sounds and using the collected data in diagnosing outcomes, according to an embodiment. Body sounds may include sounds originating from the heart, the bowel, the pharynx, the trachea, one or more large airways, one or more small airways, etc. Methodology 100 starts at step 102, in which one or more body sounds are captured using one or more sensors placed on and/or within a patient. Then at step 104, the captured body sounds are stored locally at a computing device or database suitable for storing such types of data. Then at step 106, the captured body sounds are transmitted to a remote computing device. At step 108, the captured body sounds may be analyzed manually by a user (e.g., a physician) or automatically by a computing device. The computing devices used herein may be, for example, but not limited to, a mobile phone, a tablet, a computer, a smart watch, etc.

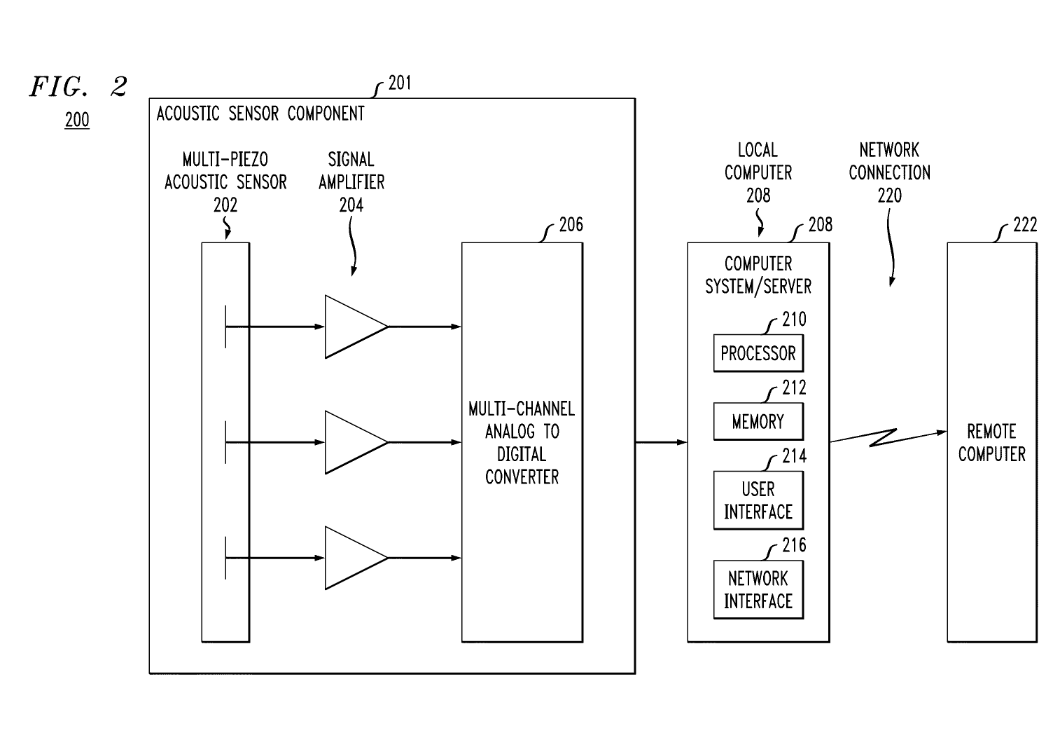

FIG. 2 depicts a system 200 for implementing methodology 100 of FIG. 1. System 200 comprises an acoustic sensor component 201, which may be a transducer that is constructed to be placed in acoustic contact with the body of the patient. This contact may be enhanced with the use of acoustic gel. The acoustic sensor component 201 comprises one or more acoustic sensors 202 (e.g., piezoelectric elements and/or piezo acoustic sensors) embedded in an acoustic matrix. The piezo elements include, but are not limited to, materials such as PZT, Kynar, quartz, etc. These materials are coated with a conductive electrode and attached to conductor wires. The piezo elements are embedded in a matrix comprising a material that includes, but is not limited to, plastics such as ABS, epoxy, polyethylene, PMMA, acrylic and polycarbonate. The piezo acoustic sensors 202 may be used in capturing one or more body sounds from a subject. A cable may connect the transducer to one or more signal amplifiers 204, where the acoustic signals generated in the piezo acoustic sensors are conditioned and connected to a multi-channel analog to digital converter 206. At this point a local computer system/server 208 stores the digital acoustic data.

Computer system/server 208 may include, but is not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like. Computer system/server 208 may include one or more processors 210 coupled to a memory 212, a user interface 214 and a network interface 216. User interface 214 may be configured to enable user input into the computer system/server 208. Network interface 216 may be configured to enable the computer system/server 208 to interface with a network and other system components.

The local computer system/server 208 may impress a time stamp on the acoustic data at the time of digitization using its internal real time clock. The digital data is periodically transmitted to a remote computer system/server 222 via a network connection 220. Network connection 220 may be a communication link comprising an internet connection, Ethernet link, local area link, cellular link, satellite link, GSM, GPRS, etc. The remote computer system/server 222 may be configured in a manner similar to the local computer system/server 208. Computer system/server 222 may comprise a cloud based server, a laptop computer, a cell phone or other suitable computing device with data storage capability. It is to be appreciated that system 200 may include more or less components than shown in FIG. 2. For example, system 200 may include multiple ones of computer system/server 208 and may also include additional components suitable for implementing methodology 100 of FIG. 1.

The system 200 may include means for analyzing and interpreting the data collected. For example, computer system/server 222 may be configured to be in communications with one or more databases comprising previously recorded data for comparison and analysis with newly captured physiological and/or pathological data.

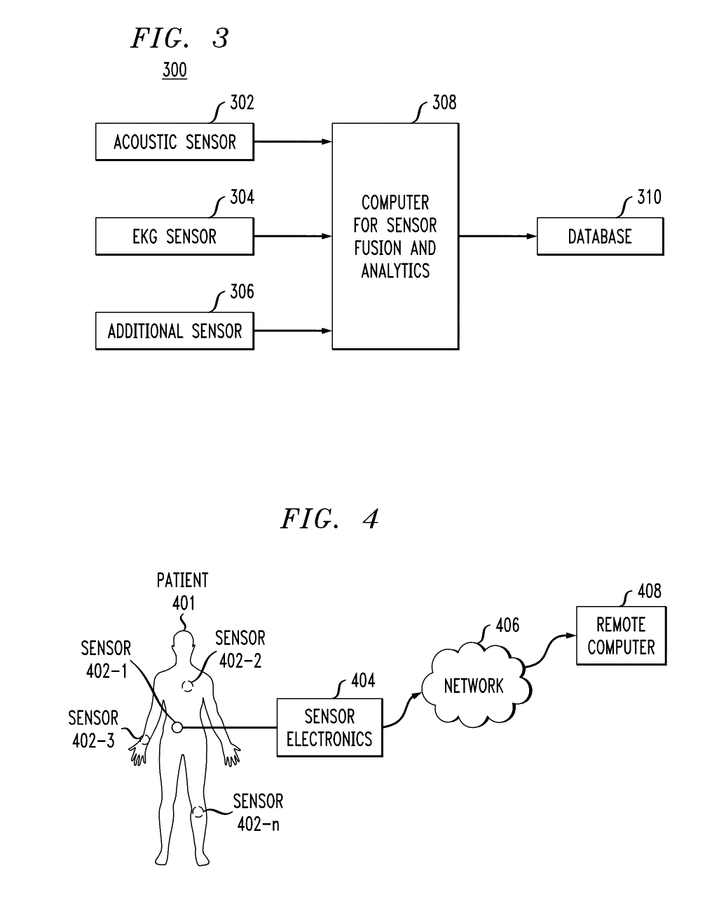

FIG. 3 illustrates an embodiment in which an acoustic sensor 302 is used in a multi-sensor context. Sensors used in this multi-sensor embodiment may include, but are not limited to, EKG sensor 304 and/or one or more additional sensors 306. Additional sensors 306 may include EEG sensors, blood pressure sensors, other acoustic sensors, etc. Physiological and/or pathological data captured using sensors 302, 304 and 306 may be sent to a local computer (e.g., local computer system/server 208) for storage and time-stamping. The time stamped acoustic data is transmitted across a network connection (e.g., network connection 220) and correlated with other time stamped sensor data in the remote computer 308. Alternatively, the captured data may be sent directly to a remote computer 308, in which the data may be stored and time-stamped. Remote computer 308 may be configured similarly to remote computer system/server 222 of FIG. 2. Remote computer 308 may be in communication with a database 310 which comprises previously recorded data (e.g., acoustic data, EKG data, and other physiological and/or pathological data) for use in analyzing newly acquired physiological and/or pathological data from sensors 302, 304 and 306. It is to be appreciated that one or more of the sensors used in embodiments of the invention may be placed on and/or placed within one or more locations of a patient.

In embodiments in which multiple sensors are used, information captured by the multiple sensors allows for more effective exclusion of non-essential information and focus on a particular area. For example, the heart has many sounds, including sounds associated with blood flow and sounds emitted by the walls of the heart. Sounds originating from the heart may be due to stenosis, aneurysm, fistula, etc. There are also sounds emitted by the walls of the heart during normal or abnormal heart function. A sound of interest may be obscured by sounds emitted concomitantly by other body compartments or systems (e.g. breathing sounds). Each abnormality would cause a different type of sound that may be differentially picked up and isolated from obscuring acoustic signals. A physician may be interested in a particular valve or a particular sound. Multiple sensor embodiments can provide the physician with means of localizing that particular sound and listening to a particular valve to accurately determine the associated conditions.

Furthermore, once the sounds are captured, sounds can be classified because the location from which the sounds originated is known and the pattern of sounds that should come from that location is also known. That is, the expected pattern of sounds may be known to an experienced physician or may be compared against known patterns stored in a database. Accordingly, any abnormal sounds can also be classified using a database and/or evaluated by a physician. Such classification may allow for the identification of yet unidentified distinct acoustic patterns associated with certain clinical conditions or that may predict the onset of a medical disorder at a future time.

In an illustrative embodiment, when dealing with heart or vascular sounds, an EKG signal can be recorded concurrently. One would know in the heart cycle when to expect to hear normal sounds, and by the timing of an abnormal sound would be better able to identify/classify the anomaly associated with this sound. The sensors can be used to capture acoustic data for the duration of the EKG signal capture. Alternatively, the sensors can be left on for as long as necessary dependent on application and physician guidance. Comparison of a recorded signal to recordings of the same subject made in the past may enable the detection of new or evolving abnormalities. Recording of time-stamped sounds using multiple sensors can allow for localizing the anatomic area of origin of the sound by comparing signal phase variation.

In some embodiments, acoustic sensors described herein may be incorporated into EKG electrodes or other types of stick-on sensors. Multiple sensors can be attached at different points of the body and measurements can be taken simultaneously at the different sites of attachment.

Embodiments described herein provide methods that allow the acoustic sensor elements described above to detect acoustic emission within the body and relate it to acoustic classifiers for use in a variety of tasks, including but not limited to: 1) suggesting diagnostic information, such as aneurysm, occlusion, narrowing, bowel obstruction, etc.; 2) enabling left-right acoustic comparisons; 3) detecting otherwise inaudible low and high frequency sounds; 4) determining the physical location of sound source(s) (i.e., spatial location estimation based on phase delay of the acoustic signal); and 5) synchronizing the acoustic information with EKG, EEG, blood pressure, and other real time measurements.

Notably, some embodiments provide systems and methods for capturing, recording, transmitting and analyzing body sound streams. As described above, some embodiments comprise at least one acoustic transducer capable of capturing audible as well as ultrasound and infrasound, an analog to digital conversion device, a microprocessor and a method of data transmission and storage both locally and at a remote server.

Auditory streams captured by the acoustic sensors (e.g., acoustic sensors 202 and 302) could be studied and compared against other patient streams and against the patient's clinical condition to allow for automated interpretation of body sounds as a diagnosis and prediction support tool. Capturing of body sounds may be carried out at the point of care or remotely using telemedicine, and can be used intermittently or can be used to monitor body sounds continuously.

In one embodiment, a device comprising a plurality of acoustic sensors may be incorporated into EKG electrodes and configured to transmit the signals they capture in conjunction with the EKG signal. Associations between the different signal types could be detected, along with associations between signals and patient known diseases, and associations between signals and current condition (e.g., echocardiographic and other imaging findings). EKG lead position can be used as an anatomic reference. In other embodiments, acoustic sensors may be used to capture sounds generated by other parts of the body (e.g., various physiological systems and locations), such as blood vessels, joints and the alimentary system.

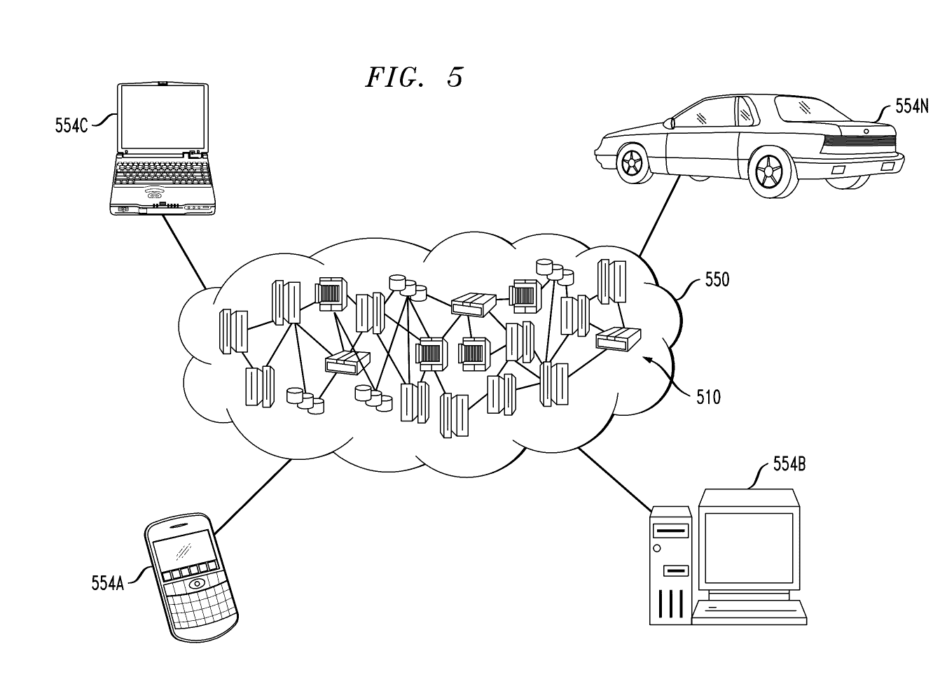

FIG. 4 illustrates an exemplary application of the system and methods described above in a patient context. As shown, an acoustic sensor 402-1 is placed in acoustic contact with patient 401 manually by a medical professional and/or affixed using stick-on retaining tape at a desired location, e.g., over the bowel in the abdomen area. Acoustic emissions from the patient 401 are captured by acoustic sensor 402-1 and relayed to sensor electronics 404, and subsequently, to a remote computer 408 using network connection 406. While not shown, the data captured by sensor electronics 404 may also be sent to a local computer, such as local computer system/server 208 of FIG. 2. Furthermore, it is to be appreciated that acoustic sensor 402-1 may be similar to the multi-piezo acoustic sensor 202, and sensor electronics 404 may comprise electronics such as signal amplifier 204 and a multi-channel analog to digital converter 206 as shown in FIG. 2. Furthermore, while acoustic sensor 402-1 and sensor electronics 404 are shown separately in FIG. 4, they may be configured to be within one device to form an acoustic component similar to acoustic sensor component 201 of FIG. 2.

Still further, it is to be appreciated that while only one acoustic sensor 402-1 is used on patient 401 in FIG. 4, multiple sensors may be used on patient 401. For example, in an alternative embodiment, one or more additional sensors 402-2 . . . 402-n shown by dotted lines (e.g., EKG sensor, blood pressure sensor, imaging device, motion sensor, respiration sensor, pulse oximetry sensor, blood pressure sensor, and other acoustics sensors) may be placed at various locations of the patient's body. In an exemplary application, a physician observes and/or listens to the aggregate of these multiple sensors 402-1 . . . 402-n, and decides that there is a sound of interest (e.g., an abnormal sound). The physician may a sensor around to get the best signal possible and have the system lock in on the sound of interest by using its phase information to localize and amplify that particular signal. After several repetitions of this manual method of localizing the sound of interest, the system and its associated algorithm eventually learns and locks onto the location where the sounds is coming from. For example, identification of the source of an acoustic signal may be performed via triangulation based on phase variation in the signal captured. The physician may enter a hypothesized condition, such as fistula, and the system may interact with a database 310 to obtain information for fistulas. The system may automatically analyze the captured sound with the stored fistula data to determine if it is indeed a fistula. Alternatively, the system may display the captured data and the stored data on a display associated with a computing device for the physician to evaluate the two signals.

In yet another embodiment, the device comprising one or more of the sensors 402-1 . . . 402-n is placed at one or more locations on/in the patient. The device consults a database and picks potential candidate conditions and uses that to lock up on the locations. A physician may manually select a sound of interest or the device may interact with the database again to narrow down the list of candidates. The system then asks the physician if this is what the physician is interested in, if not, the system moves to next the sound and presents all the candidates to the physician one at a time.

Advantageously, various embodiments described herein may utilize one or more acoustic sensor elements to perform one or more of the following tasks:
Detect acoustic emission within the body and relate it to acoustic classifiers to suggest diagnostic information including but not limited to aneurysm, occlusion, narrowing, bowel obstruction, etc.
Enable left—right acoustic comparison in, for example but not limited to, femoral arteries, carotid arteries, relative air flow in lungs, etc.
Support long duration monitoring, e.g., continuous monitoring for a period of time that would be impractical at a physician's office. For example, more than an hour, over the course of a day or week, etc.
Detect otherwise inaudible low and high frequency sounds.
Determine the physical location of sound source(s) using, by way of example but not limited to, spatial location estimation based on phase delay of the acoustic signal.
Synchronize acoustic information with EKG, EEG, blood pressure and other real time measurements.
Enable integration with other sensors including but not limited to an EKG electrode.
Capture sounds originating from blood flow.
Capture sounds generated by the movement of joints and bones.
Capture sounds generated by friction between planes, such as sounds generated by the pericardium or pleurae.

Embodiments of the present invention may be a system, a method, and/or a computer program product at any possible technical detail level of integration. The computer program product may include a computer readable storage medium (or media) having computer readable program instructions thereon for causing a processor to carry out aspects of the present invention. For example, computer system/server 208 may comprise a computer program product for implementing embodiments of the invention disclosed herein.

The computer readable storage medium (e.g., memory 212) can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network (e.g., network 220, 406), including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is understood in advance that although this disclosure includes a detailed description on cloud computing below, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.
Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

Computer system/server 208 and 222 in FIG. 2, computer system 308 in FIG. 3 and computer system 408 in FIG. 4 are examples of cloud computing nodes. It is to be appreciated, however, that these computer systems/servers are only examples of suitable cloud computing nodes and is not intended to suggest any limitation as to the scope of use or functionality of embodiments of the invention described herein. Regardless, these computer systems/servers are examples of cloud computing nodes capable of being implemented and/or performing any of the functionality set forth hereinabove.

Referring now to FIG. 5, illustrative cloud computing environment 550 is depicted. As shown, cloud computing environment 550 comprises one or more cloud computing nodes 510 with which local computing devices used by cloud consumers, such as, for example, personal digital assistant (PDA) or cellular telephone 554A, desktop computer 554B, laptop computer 554C, and/or automobile computer system 554N may communicate. Nodes 510 may communicate with one another. They may be grouped (not shown) physically or virtually, in one or more networks, such as Private, Community, Public, or Hybrid clouds as described hereinabove, or a combination thereof. This allows cloud computing environment 550 to offer infrastructure, platforms and/or software as services for which a cloud consumer does not need to maintain resources on a local computing device. It is understood that the types of computing devices 554A-N shown in FIG. 5 are intended to be illustrative only and that computing nodes 510 and cloud computing environment 550 can communicate with any type of computerized device over any type of network and/or network addressable connection (e.g., using a web browser).

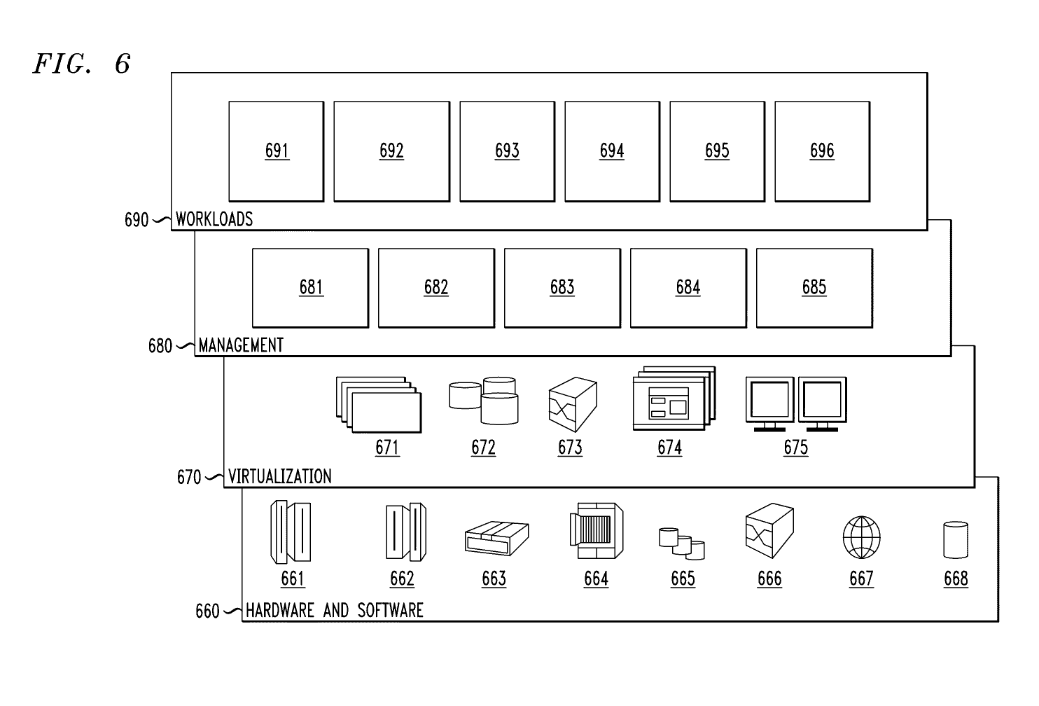

Referring now to FIG. 6, a set of functional abstraction layers provided by cloud computing environment 550 (FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 660 includes hardware and software components. Examples of hardware components include: mainframes 661; RISC (Reduced Instruction Set Computer) architecture based servers 662; servers 663; blade servers 664; storage devices 665; and networks and networking components 666. In some embodiments, software components include network application server software 667 and database software 668.

Virtualization layer 670 provides an abstraction layer from which the following examples of virtual entities may be provided: virtual servers 671; virtual storage 672; virtual networks 673, including virtual private networks; virtual applications and operating systems 674; and virtual clients 675.

In one example, management layer 680 may provide the functions described below. Resource provisioning 681 provides dynamic procurement of computing resources and other resources that are utilized to perform tasks within the cloud computing environment. Metering and Pricing 682 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 683 provides access to the cloud computing environment for consumers and system administrators. Service level management 684 provides cloud computing resource allocation and management such that required service levels are met. Service Level Agreement (SLA) planning and fulfillment 685 provide pre-arrangement for, and procurement of, cloud computing resources for which a future requirement is anticipated in accordance with an SLA.

Workloads layer 690 provides examples of functionality for which the cloud computing environment may be utilized. Examples of workloads and functions which may be provided from this layer include: mapping and navigation 691; software development and lifecycle management 692; virtual classroom education delivery 693; data analytics processing 694; transaction processing 695; and object detection and tracking 696, which may implement the functionality described above with respect to FIGS. 1-5.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
capturing one or more body sounds using one or more acoustic sensors placed at one or more locations of a subject;
converting the one or more body sounds into one or more acoustic signals using one or more acoustic transducers;
transmitting the one or more acoustic signals to one or more computing devices;
comparing the one or more acoustic signals against one or more other signals; and
identifying at least one of one or more physiological conditions and one or more pathological conditions based on the comparison;
wherein the capturing, converting, transmitting, comparing and identifying steps are performed by at least one processor device coupled to a memory.

2. The method of claim 1, further comprising capturing one or more additional signals from the subject using one or more additional sensors placed at one or more additional locations of the subject.

3. The method of claim 2, wherein the one or more additional sensors comprise at least one of an imaging device, an electrocardiogram sensor, an electroencephalogram sensor, a motion sensor, a respiration sensor, a pulse oximetry sensor, and a blood pressure sensor.

4. The method of claim 2, wherein the one or more additional signals are captured at the same time of capturing the one or more body sounds.

5. The method of claim 2, further comprising synchronizing the one or more acoustic signals with the one or more additional signals.

6. The method of claim 5, further comprising comparing the acoustic signals with the one or more additional signals.

7. The method of claim 1, wherein the one or more other signals comprise signals obtained from a database.

8. The method of claim 7, wherein the signals obtained from the database comprise signals previously obtained from one or more other subjects.

9. The method of claim 7, wherein the signals obtained from the database comprises signals previously obtained from the subject.

10. The method of claim 1, further comprising amplifying at least one of the one or more acoustic signals.

11. The method of claim 1, further comprising localizing a source of one of the one or more acoustic signals by triangulation based on phase information associated with the one or more acoustic signals.

12. The method of claim 1, wherein the one or more acoustic sensors comprises piezo acoustic sensors.

13. The method of claim 1, wherein the one or more body sounds comprise one or more of heart sounds, vascular murmurs and bruits.

14. The method of claim 1, wherein the one or more body sounds comprise sounds originating from at least one of a pharynx, a trachea, one or more large airways, and one or more small airways.

15. The method of claim 1, wherein the one or more body sounds comprise bowel sounds generated by one or more portions of a gastrointestinal tract.

16. The method of claim 1, wherein the one or more acoustic sensors are secured to the subject for a prolonged period of time.

17. The method of claim 1, wherein identification of at least one of the one or more physiological conditions and one or more pathological conditions comprises displaying the comparison to a user.

18. The method of claim 1, wherein identification of at least one of the one or more physiological conditions and one or more pathological conditions is performed automatically by an algorithm implemented by the at least one processor device.

19. A device comprising:
one or more acoustic sensors;
one or more transducers; and
a memory and a processor operatively coupled to the memory and configured to implement the steps of:
capturing one or more body sounds using the one or more acoustic sensors placed at one or more locations of a subject;
converting the one or more body sounds into one or more acoustic signals using the one or more acoustic transducers;
transmitting the one or more acoustic signals to one or more computing devices;
comparing the one or more acoustic signals against one or more other signals; and
identifying at least one of one or more physiological conditions and one or more pathological conditions based on the comparison.

20. A computer program product comprising a computer readable storage medium for storing computer readable program code which, when executed, causes a computer to:
capture one or more body sounds using one or more acoustic sensors placed at one or more locations of a subject;
convert the one or more body sounds into one or more acoustic signals using one or more acoustic transducers;
transmit the one or more acoustic signals to one or more computing devices;
compare the one or more acoustic signals against one or more other signals; and
identify at least one of one or more physiological conditions and one or more pathological conditions based on the comparison.
